(19) United States
(12) Patent Application Publication
(10) Pub. No.: US 2020/0090795 A1
(43) Pub. Date: Mar. 19, 2020

(54) METHOD AND SYSTEM FOR SHARING PRIVACY DATA BASED ON SMART CONTRACTS

(71) Applicant: HTC Corporation, Taoyuan City (TW)

(72) Inventors: Edward Chang, Taoyuan City (TW); Chung-Huan Mei, Taoyuan City (TW); Chun-Ting Liu, Taoyuan City (TW); Shih-Wei Liao, Taoyuan City (TW); Wei-Chen Lin, Taoyuan City (TW); Pin-Wei Liao, Taoyuan City (TW); Wei-Kang Fu, Taoyuan City (TW)

(73) Assignee: HTC Corporation, Taoyuan City (TW)

(21) Appl. No.: 16/568,254

(22) Filed: Sep. 12, 2019

Related U.S. Application Data
(60) Provisional application No. 62/731,105, filed on Sep. 14, 2018.

(51) Int. Cl. G16H 10/60; H04L 9/06; G06F 21/62; G06F 21/55; H04L 29/06

(52) U.S. Cl. CPC G16H 10/60; H04L 9/0637; G06F 21/6245; G06F 2221/034; H04L 63/102; H04L 2209/38; G06F 21/552

(57) ABSTRACT

The present disclosure provides a method and system for sharing privacy data based on smart contracts. The method includes: receiving, from a first providing device, a first pointer directing to a data contract, and adding the first pointer to a relationship contract of a first user, where the data contract is deployed on a first blockchain by the first providing device in response to the newly added first privacy data, and the first privacy data belongs to the first user; in response to the first privacy data being authorized to be shared with a second providing device, providing the first pointer to the second providing device to allow the second providing device to access the first privacy data through the first pointer.

### CROSS-REFERENCE TO RELATED APPLICATION

This application claims the priority benefit of U.S. provisional application Ser. No. 62/731,105, filed on Sep. 14, 2018. The entirety of the above-mentioned patent application is hereby incorporated by reference herein and made a part of this specification.

### BACKGROUND OF THE DISCLOSURE

### Field of the Disclosure

The present disclosure relates to a blockchain technology, and in particular, to a method and system for sharing privacy data based on smart contracts.

### Description of Related Art

A new data organization structure that is referred to as a blockchain and an operation method are booming with the disclosure of digital cryptocurrency. The blockchain can be distributed, decentralized, and programmable with high security and reliability, of which data can be collectively maintained and shared. In addition to the field of digital cryptocurrency, the blockchain technology is also quite suitable for fields such as data encryption storage, financial transactions, Internet of Things, and the like. Even some scholars predict that blockchain technology will be a cornerstone of the next generation Internet, which will lead the development of the future network.

At present, the blockchain technology has attracted a lot of attention from many fields represented by finance and the Internet, governments, and capital markets. Nowadays, the blockchain technology has been gradually applied to development of fields such as financial transactions, e-government, and the like, but has not been applied to the field of privacy protection. With the development of a network hacking technology, public privacy is facing great threats, and the blockchain technology strengthens privacy data protection. However, how to promote integration of the blockchain technology and privacy protection requirements is a problem to be urgently resolved.

### SUMMARY OF THE DISCLOSURE

In view of this, the present disclosure provides a method and system for sharing privacy data based on smart contracts, which can be used to resolve the foregoing technical problems.

The present disclosure provides a method for sharing privacy data based on smart contracts, including: in response to a first providing device adding first privacy data associated with a first user, deploying, by the first providing device, a data contract on a first blockchain, where the first privacy data is stored in a first local database of the first providing device and includes first metadata, and the data contract records the first metadata of the first privacy data; adding, by the first providing device, a first pointer to a relationship contract of the first providing device, and providing the first pointer to a first electronic device controlled by the first user, where the first pointer directs to the data contract; adding, by the first electronic device, a first pointer to a relationship contract of the first user; in response to the first privacy data being authorized to be shared with a second providing device, providing, by the first electronic device, the first pointer to the second providing device to allow the second providing device to access the first privacy data through the first pointer.

The present disclosure provides a system for sharing privacy data based on smart contracts, including a first providing device, a second providing device, and a first electronic device. The first electronic device is controlled by a first user. In response to a first providing device adding first privacy data associated with the first user, the first providing device deploys a data contract on a first blockchain, where the first privacy data is stored in a first local database of the first providing device and includes first metadata, and the data contract records the first metadata of the first privacy data; the first providing device adds a first pointer to a relationship contract of the first providing device, and provides the first pointer to a first electronic device controlled by the first user, where the first pointer directs to the data contract; the first electronic device adds a first pointer to a relationship contract of the first user; in response to the first privacy data being authorized to be shared with a second providing device, the first electronic device provides the first pointer to the second providing device to allow the second providing device to access the first privacy data through the first pointer.

The present disclosure provides a method for sharing privacy data based on smart contracts adapted for a first electronic device held by a first user, the method including: receiving a first pointer directing to a data contract from a first providing device, and adding a first pointer to a relationship contract of the first user, where the data contract is deployed on a first blockchain by the first providing device in response to the newly added first privacy data, and the first privacy data belongs to the first user; and in response to the first privacy data being authorized to be shared with a second providing device, providing the first pointer to the second providing device to allow the second providing device to access the first privacy data through the first pointer.

Based on the above, according to the method and system of the present disclosure, a user can select privacy data and an object that are to be shared. Moreover, because the data shared by the user is forwarded through the electronic device held by the user, it is not necessary to use additional manpower and money to construct a specific interface structure between respective providing devices, thereby enhancing convenience of sharing data while reducing associated costs in manpower and money.

To make the aforementioned features and advantages of the present disclosure more comprehensible, embodiments accompanied with figures are described in detail below.

### BRIEF DESCRIPTION OF THE DRAWINGS

FIG. 1 is a schematic diagram of a system for sharing privacy data based on smart contracts according to an embodiment of the present disclosure.

FIG. 2 is a flowchart of a method for sharing privacy data based on smart contracts according to an embodiment of the present disclosure.

FIG. 3 is a schematic diagram of a service architecture according to an embodiment of the present disclosure.

FIG. 4 is a flowchart of a method for sharing privacy data based on smart contracts according to an embodiment of the present disclosure.

### DESCRIPTION OF THE EMBODIMENTS

Briefly, according to a method and system of the present disclosure, a user can securely share privacy data thereof to different objects based on smart contracts in a blockchain technology. For convenience of description, in the following, it is assumed that the privacy data is case history data of the user, but the present disclosure may also be applicable to other types of privacy data and is not limited thereto. In addition, various contracts mentioned below are all stylized specific smart contracts.

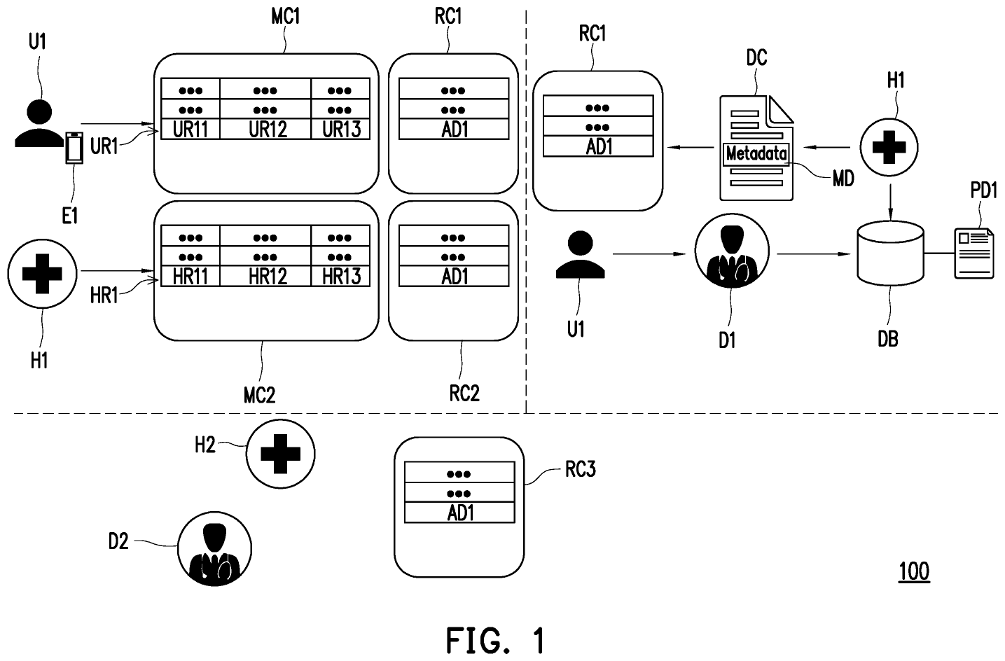

Referring to FIG. 1, FIG. 1 is a schematic diagram of a system for sharing privacy data based on smart contracts according to an embodiment of the present disclosure. As shown in FIG. 1, a system 100 includes a first electronic device E1 and a first providing device H1. In different embodiments, the first electronic device E1 may be controlled by a first user U1, and may be implemented as various smart devices (such as smart phones, tablets, etc.) and computer devices (such as notebook computers, personal computer, etc.), but is not limited thereto. The first providing device H1 is, for example, a data management system corresponding to a medical location (for example, various hospitals and clinics), which may be configured to store various case history data recorded by medical personnel. For convenience of description, in the following, it is assumed that the first providing device H1 corresponds to a data management system of a hospital A, but the present disclosure is not limited thereto.

In an embodiment, if the first user U1 is to use services of the present disclosure, the first user U1 may perform, through the first electronic device E1, a user registration operation to a management system maintained by a related competent authority (for example, Taiwan Centers for Disease Control). Correspondingly, the management system of the competent authority may deploy a relationship contract RC1 associated with the first user U1 on a first blockchain. In different embodiments, the relationship contract RC1 may be used to store one or more pointers, and these pointers may be individually used to access different privacy data (for example, case history data) that belongs to the first user U1.

In different embodiments, the first blockchain may be implemented based on any known blockchain technology, but is not limited thereto. In addition, in some embodiments, the first blockchain may further be used as a branch chain of a second blockchain (for example, an Ethereum public blockchain), of which relevant details are to be described later.

In addition, the management system of the competent authority may pre-deploy a user member contract MC1 on the first blockchain, which can be used to record user identity records of different users. Therefore, in response to the user registration operation of the first user U1, the management system may correspondingly add, to the user member contract MC1, a user identity record UR1 corresponding to the first user U1. In this embodiment, the user identity record UR1 may include an identity hash value UR11, a blockchain address UR12, and a first relationship contract address UR13 of the first user U1, where the first relationship contract address UR13 directs to the relationship contract RC1 of the first user U1, and the identity hash value UR11 is generated based on identity information (for example, a name, an identity card number, etc., but being not limited thereto) of the first user U1.

Furthermore, if the first providing device H1 also intends to use the service proposed by the present disclosure, the first providing device H1 may perform a provider registration operation to the management system. Correspondingly, the management system of the competent authority may deploy a relationship contract RC2 associated with the first providing device H1 on a first blockchain. In different embodiments, the relationship contract RC2 may be used to store one or more pointers, and these pointers may be used to access privacy data (for example, case history data) that belongs to one or more users.

In addition, the management system of the competent authority may pre-deploy a provider member contract MC2 on the first blockchain, which can be used to record provider identity records of different providing devices. Therefore, in response to the provider registration operation of the first providing device H1, the management system may correspondingly add, to the provider member contract MC2, a provider identity record HR1 corresponding to the first providing device H1. In this embodiment, the provider identity record HR1 may include an identification number HR11 (for example, a number of a hospital A) of the first providing device H1, a name HR12 (for example, the hospital A), and a second relationship contract address HR13, where the second relationship contract address HR13 directs to the relationship contract RC2 of the first providing device H1.

It should be understood that stakeholders of the first blockchain (which may be regarded as a public ledger) are notified, in the form of a transaction, of various operations of deploying and modifying smart contracts mentioned in the present disclosure.

In an embodiment, after the first user U1 is diagnosed by a doctor D1 in the hospital A, the doctor D1 may create a current inspection record to be associated with first privacy data PD1 (that is, case history data) of the first user U1, and the first providing device H1 may add the first privacy data PD1 to a local database DB of the first providing device H1. Then, the first user U1 may share the first privacy data PD1 to other providing devices (for example, data management systems of other hospitals) through the method proposed by the present disclosure. Further description is provided below.

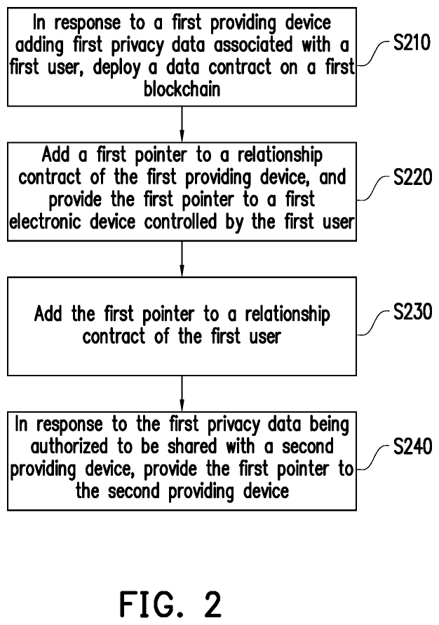

Referring to FIG. 2, FIG. 2 is a flowchart of a method for sharing privacy data based on smart contracts according to an embodiment of the present disclosure. The method of this embodiment may be performed by the system 100 of FIG. 1, and details of the steps of FIG. 2 are described with reference to the content shown in FIG. 1 below.

First, in step S210, in response to a first providing device H1 adding first privacy data PD1 associated with the first user U1, the first providing device H1 may deploy a data contract DC on a first blockchain. In this embodiment, the first privacy data PD1 is stored in a local database DB of the first providing device H1 and includes first metadata MD, and the first metadata MD may be recorded in the data contract DC.

In different embodiments, the first metadata MD may include at least one of a name (for example, a name of a hospital A) of the first providing device H1, a diagnostic department and a diagnostician D1 that generate case history data, and a diagnosis time of the case history data, but is not limited thereto.

In an embodiment, the first metadata MD may further include a hash value field, which may record a data hash value, where the data hash value is generated based on the first privacy data PD1. Therefore, if any device attempts to access the first privacy data PD1 in the future, it may be verified, based on the data hash value, whether the first privacy data PD1 is falsified, thereby ensuring correctness of the data.

In another embodiment, the first metadata MD may further include an access permission field, which may record a name of a device that may access the first privacy data PD1. Because the first privacy data PD1 belongs to a first user U1 and is added by the first providing device H1, the access permission field in the first metadata MD may record the name of the first providing device H1 and a name of the first user U1, to indicate that the first providing device H1 and the first user U1 have the right to access the first privacy data PD1, which may be not limited thereto.

Then, in step S220, the first providing device H1 may add a first pointer AD1 to a relationship contract RC2 of the first providing device H1, and provides the first pointer AD1 to a first electronic device E1 controlled by the first user U1. In this embodiment, the first pointer AD1 directs to the data contract DC.

In addition, in step S230, the first electronic device E1 may add the first pointer AD1 to a relationship contract RC1 of the first user.

In an embodiment, the first electronic device E1 may provide a user interface corresponding to a service of the present disclosure. In this case, after the first pointer AD1 is added to the first electronic device E1, the user interface may correspondingly obtain the first metadata MD of the first privacy data PD1 based on the first pointer AD1. For example, the metadata MD (for example, the hospital A associated with the first privacy data PD1, the physician D1, the diagnosis time, the diagnosis department, etc.) may be presented on the user interface for reference by the first user U1, but is not limited thereto.

In addition, the user interface may also provide metadata of other privacy data for reference by the first user U1. In this case, the first user U1 may select, in the user interface, one or more pieces of privacy data and an object that are to be shared.

For example, assuming that the first user U1 is to share the first privacy data PD1 to a second providing device H2 (which corresponds to, for example, a data management system of a hospital B), the first user U1 may select the second providing device H2 again as the object to share the first privacy data PD1 after selecting the first privacy data PD1. In an embodiment, the user interface may be selected by the first user U1 in a manner that a plurality of providing devices are selected in a pull-down menu, but the present disclosure is not limited thereto.

In different embodiments, each of the providing devices (for example, the second providing device H2) in the pull-down menu may be registered in advance on the management system of the competent authority via a corresponding provider registration operation. That is, provider identity records of the providing devices may be recorded in a provider member contract maintained by the management system.

In an embodiment, in response to the first user U1 selecting the first privacy data PD1 in the user interface of the first electronic device E1 and correspondingly selecting, as an object to share the first privacy data PD1, the second providing device H2 from the plurality of providing devices displayed on the user interface, the first electronic device E1 may determine that the first privacy data PD1 is authorized to be shared with the second providing device H2.

Afterwards, in step S240, in response to the first privacy data PD1 being authorized to be shared with the second providing device H2, the first electronic device E1 may provide the first pointer AD1 to the second providing device H2 to allow the second providing device H2 to access the first privacy data PD1 through the first pointer AD1. In an embodiment, the second providing device H2 may add the first pointer AD1 to a relationship contract RC3 of the second providing device H2.

In this case, the physician D2 located in the hospital B can obtain the metadata MD of the first privacy data PD1 through the first pointer AD1 recorded in the relationship contract RC3 of the second providing device H2. In addition, in an embodiment, the physician D2 (or other person who can use the second providing device H2) may attempt to access the content (for example, diagnostic content, a prescription, etc.) of the first privacy data PD1 by triggering the first pointer AD1.

In an embodiment, in response to the first pointer AD1 in the relationship contract RC3 of the second providing device H2 being triggered, the second providing device H2 may send an access request for accessing the first privacy data PD1 to the first electronic device E1. Afterwards, the first electronic device E1 may forward the access request to the first providing device H1.

Correspondingly, the first providing device H1 may perform verification operation on at least one of the second providing device H2 and the first privacy data PD1. In an embodiment, assuming that the metadata MD includes an access permission field, in response to the first privacy data PD1 being authorized to be shared with the second providing device H2, the first providing device H1 may add a name of the second providing device H2 to an access permission field of the metadata MD.

In this case, if the first providing device H1 receives an access request for accessing the first privacy data PD1 of the second providing device H2 from the first electronic device E1, the first providing device H1 may determine whether the name of the second providing device H2 exists in the access permission field of the metadata MD. If yes, the first providing device H1 may determine that the second providing device H2 is a legal accessor of the first privacy data PD1, and correspondingly determine that the second providing device H2 has been verified. Conversely, if the name of the second providing device H2 does not exist in the access permission field of the metadata MD, the first providing device H1 may determine that the second providing device H2 is not a legal accessor of the first privacy data PD1, and correspondingly determine that the second providing device H2 has not been verified, but the present disclosure is not limited thereto.

After it is determined that the second providing device H2 passes the verification operation, the first providing device H1 may provide the first privacy data PD1 in the local database DB to the second providing device H2 through the first electronic device E1. Specifically, the first providing device H1 may forward the first privacy data PD1 to the first electronic device E1, and the first electronic device E1 may forward the first privacy data PD1 to the second providing device H2 again.

In an embodiment, if the metadata MD includes a hash value field (which records a data hash value), after the second providing device H2 obtains the first privacy data PD1 from the first electronic device E1, a reference hash value may be calculated based on the first privacy data PD1. In response to the reference hash value matching the data hash value, the second providing device H2 may determine that the first privacy data PD1 is not falsified. Conversely, if the reference hash value does not match the data hash value, the second providing device H2 may determine that the first privacy data PD1 has been falsified. Accordingly, the second providing device H2 may further verify the correctness of the first privacy data PD1, thereby preventing the relevant person (for example, the physician D2) from referring to the falsified data.

Through the foregoing mechanism, the physician D2 may find, in the hospital B, the past case history data (that is, the first privacy data PD1) of the first user U1 in the hospital A as a reference for the current diagnosis.

Furthermore, although the hospital B is used as an example of the second providing device H2 for description above, the embodiment of the present disclosure is not limited thereto. In other embodiments, the second providing device H2 may also correspond to an insurer, an agent (for example, a lawyer, a family member, etc.) of the first user U1, or any entity that the first user U1 is to authorize for accessing privacy data thereof. For example, if the second providing device H2 corresponds to an insurer, the first user U1 may authorize the insurer to access case history data (that is, privacy data) thereof via the service of the present disclosure, so as to facilitate the insurer for claims or other related procedures. In addition, if the second providing device H2 corresponds to the agent of the first user U1, when the first user U1 loses behaviour ability, the agent may also obtain the case history data (that is, privacy data) of the first user U1 via the service of the present disclosure, so as to handle medical procedures or other required processing. This is not limited thereto.

Based on the above, according to the method proposed through the present disclosure, the user can select the privacy data and the object that are to be shared. Moreover, because the data shared by the user is forwarded through the electronic device held by the user, it is not necessary to use additional manpower and money to construct a specific interface structure between respective providing devices, thereby enhancing convenience of sharing data while reducing associated costs in manpower and money. In this way, accountability and transparency, ownership and accessibility when the privacy data is shared can be achieved.

Moreover, because the first blockchain (that is, a public account book) stores only the pointer directing to the data contract, and does not store actual privacy data, privacy compliance can be achieved.

Carrying on with the previous embodiments, each of the operations of deploying and modifying smart contracts mentioned in the present disclosure is notified of stakeholders of the first blockchain as a transaction. According to the operation principle of the blockchain technology, these transactions are finally packaged into blocks and exist in the first blockchain.

In an embodiment, according to the present disclosure, a branch chain hash value may further be generated based on a plurality of blocks on the first blockchain regularly or irregularly, and the branch chain hash value is used as a transaction to be released to the second blockchain (for example, an Ethereum public blockchain). In general, a consensus generation mechanism of the Ethereum public blockchain is less efficient, and under the framework of the present disclosure, because the relevant stakeholders of the first blockchain may adopt a relatively efficient consensus generation method, a better transaction throughput can be obtained. Moreover, in a manner of recording the branch chain hash value in the second blockchain, the correctness of various types of data on the first blockchain may further be ensured.

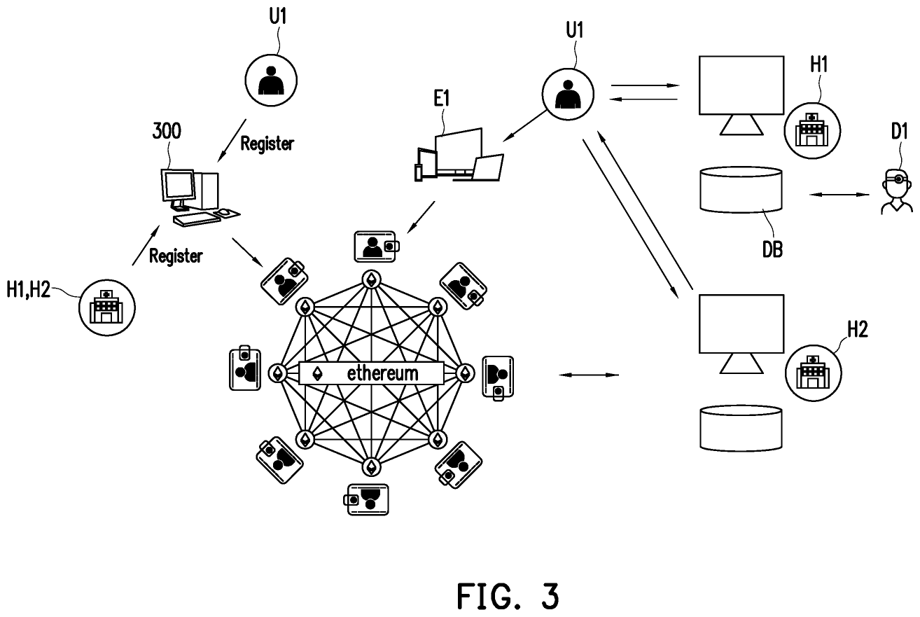

Referring to FIG. 3, FIG. 3 is a schematic diagram of a service architecture according to an embodiment of the present disclosure. In this embodiment, a first user U1, a first providing device H1, and a second providing device H2 may perform corresponding registration operations to a management system 300 of the relevant competent authority. After that, the first user U1 may use a first electronic device E1 to share privacy data between the first providing device H1 and the second providing device H2. In addition, when an Ethereum public blockchain is used as a second blockchain, blocks on a first blockchain may further be released as a transaction to the second block after being calculated as the branch chain hash value. For related details, reference may be made to the description in the previous embodiment, and the descriptions thereof are omitted herein.

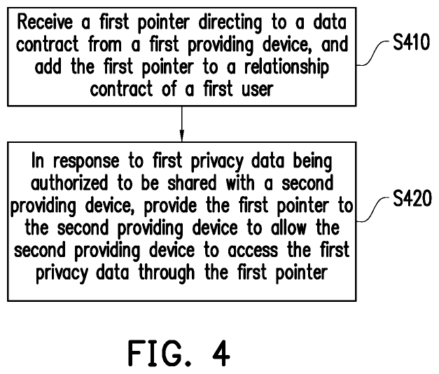

Referring to FIG. 4, FIG. 4 is a flowchart of a method for sharing privacy data based on smart contracts according to an embodiment of the present disclosure. The method of this embodiment may be performed by a first electronic device E1 of FIG. 1, and details of the steps of FIG. 4 are described with reference to the content shown in FIG. 1 below.

First, in step S410, the first electronic device E1 may receive a first pointer AD1 directing to a data contract DC from a first providing device H1, and add the first pointer AD1 to a relationship contract RC1 of a first user U1. Afterwards, in step S420, in response to first privacy data PD1 being authorized to be shared with a second providing device H2, the first electronic device E1 may provide the first pointer AD1 to the second providing device H2 to allow the second providing device H2 to access the first privacy data PD1 through the first pointer AD1. For details of steps in FIG. 4, reference may be made to the description in the previous embodiment, and the descriptions thereof are omitted herein.

From the above, according to the method and system of the present disclosure, the user can select privacy data and an object that are to be shared. Moreover, because the data shared by the user is forwarded through the electronic device held by the user, it is not necessary to use additional manpower and money to construct a specific interface structure between respective providing devices, thereby enhancing convenience of sharing data while reducing associated costs in manpower and money. In this way, accountability and transparency, ownership and accessibility when the privacy data is shared can be achieved. Moreover, because the first blockchain (that is, a public account book) stores only the pointer directing to the data contract, and does not store actual privacy data, privacy compliance can be achieved.

In addition, according to the present disclosure, a branch chain hash value may further be generated based on a plurality of blocks on the first blockchain regularly or irregularly, and the branch chain hash value is used as a transaction to be released to the second blockchain (for example, the Ethereum public blockchain), and therefore a better transaction throughput can be obtained. Moreover, in a manner of recording the branch chain hash value in the second blockchain, the correctness of various types of data on the first blockchain may further be ensured.

Although the disclosure is described with reference to the above embodiments, the embodiments are not intended to limit the disclosure. A person of ordinary skill in the art may make variations and modifications without departing from the spirit and scope of the disclosure. Therefore, the protection scope of the disclosure should be subject to the appended claims.

What is claimed is:

1. A method for sharing privacy data based on smart contracts, comprising:
in response to a first providing device adding a first privacy data associated with a first user, deploying, by the first providing device, a data contract on a first blockchain, wherein the first privacy data is stored in a first local database of the first providing device and has a first metadata, and the data contract records the first metadata of the first privacy data;
adding, by the first providing device, a first pointer to a relationship contract of the first providing device, and providing the first pointer to a first electronic device controlled by the first user, wherein the first pointer directs to the data contract;
adding, by the first electronic device, the first pointer to a relationship contract of the first user; and
in response to the first privacy data being authorized to be shared with a second providing device, providing, by the first electronic device, the first pointer to the second providing device to allow the second providing device to access the first privacy data through the first pointer.

2. The method according to claim 1, wherein the first privacy data is a case history data of the first user, the first providing device corresponds to a first medical location, and the second providing device corresponds to at least one of a second medical location, an insurer, and an agent of the first user.

3. The method according to claim 2, wherein the first metadata of the first privacy data comprises at least one of a name of the first providing device, a diagnostic department and a diagnostician that generate the case history data, and a diagnosis time of the case history data.

4. The method according to claim 1, further comprising:
adding, by the second providing device, the first pointer to a relationship contract of the second providing device; and
in response to the first pointer in the relationship contract of the second providing device being triggered, sending, by the second providing device, an access request for accessing the first privacy data to the first electronic device.

5. The method according to claim 1, wherein the first metadata of the first privacy data comprises a hash value field in which a data hash value is recorded, and the data hash value is generated based on the first privacy data.

6. The method according to claim 5, further comprising:
in response to the second providing device obtaining the first privacy data, calculating, by the second providing device, a reference hash value based on the first privacy data; and
in response to the reference hash value matching the data hash value, determining, by the second providing device, that the first privacy data is not tampered with; otherwise, determining that the first privacy data has been tampered with.

7. The method according to claim 1, wherein the first metadata of the first privacy data comprises an access permission field, in which a name of the first providing device is recorded, and in response to the first privacy data being authorized to be shared with the second providing device, the method further comprises:
adding, by the first providing device, a name of the second providing device to the access permission field.

8. The method according to claim 7, further comprising:
in response to receiving an access request of the second providing device for accessing the first privacy data, determining whether the name of the second providing device exists in the access permission field; and
in response to the name of the second providing device existing in the access permission field, determining that the second providing device is a legal accessor of the first privacy data; otherwise, determining that the second providing device is not a legal accessor of the first privacy data.

9. The method according to claim 1, wherein in response to the first providing device adding the first privacy data associated with the first user, before the step of generating a first transaction, the method further comprises:
deploying, in response to a user registration operation of the first user, the relationship contract associated with the first user on the first blockchain; and
adding a user identity record associated with the first user to a user member contract on the first blockchain, wherein the user identity record comprises an identity hash value of the first user, a blockchain address, and a first relationship contract address, wherein the first relationship contract address directs to the relationship contract of the first user, and the identity hash value is generated based on identity information of the first user.

10. The method according to claim 1, wherein in response to the first providing device adding the first privacy data associated with the first user, before the step of generating a first transaction, the method further comprises:
deploying, in response to a provider registration operation of the first providing device, the relationship contract associated with the first providing device on the first blockchain; and
adding a provider identity record associated with the first providing device to a provider member contract on the first blockchain, wherein the provider identity record comprises an identification number, a name, and a second relationship contract address of the first providing device, and the second relationship contract address directs to the relationship contract of the first providing device.

11. The method according to claim 1, wherein in response to the first user selecting the first privacy data in a user interface of the first electronic device and correspondingly selecting, as an object to share the first privacy data, the second providing device from a plurality of providing devices displayed on the user interface, it is determined that the first privacy data is authorized to be shared with the second providing device.

12. The method according to claim 1, further comprising:
in response to receiving an access request of the second providing device for accessing the first privacy data from the first electronic device, performing a verification operation on the second providing device; and
in response to the second providing device passing the verification operation, providing, by the first providing device, the first privacy data in the first local database to the second providing device through the first electronic device.

13. The method according to claim 12, wherein the step of providing the first privacy data in the first local database to the second providing device through the first electronic device comprises:
forwarding, by the first providing device, the first privacy data to the first electronic device held by the first user; and
forwarding, by the first electronic device, the first privacy data to the second providing device.

14. The method according to claim 1, further comprising:
generating a chain hash value based on a plurality of blocks on the first blockchain; and
releasing the chain hash value as a transaction to a second blockchain.

15. The method according to claim 14, wherein the second blockchain is an Ethereum public blockchain.

16. A system for sharing privacy data based on smart contracts, comprising:
a first providing device;
a first electronic device controlled by a first user, wherein
in response to the first providing device adding a first privacy data associated with the first user, the first providing device deploys a data contract on a first blockchain, wherein the first privacy data is stored in a first local database of the first providing device and has a first metadata, and the data contract records the first metadata of the first privacy data;
the first providing device adds a first pointer to a relationship contract of the first providing device and provides the first pointer to the first electronic device, and the first pointer directs to the data contract;
the first electronic device adds the first pointer to a relationship contract of the first user; and
in response to the first privacy data being authorized to be shared with a second providing device, the first electronic device provides the first pointer to the second providing device to allow the second providing device to access the first privacy data through the first pointer.

17. A method for sharing privacy data based on smart contracts, adapted to a first electronic device held by a first user and comprising:
receiving, from a first providing device, a first pointer directing to a data contract, and adding the first pointer to a relationship contract of the first user, wherein the data contract is deployed on a first blockchain by the first providing device in response to a newly added first privacy data, and the first privacy data belongs to the first user;
in response to the first privacy data being authorized to be shared with a second providing device, providing the first pointer to the second providing device to allow the second providing device to access the first privacy data through the first pointer.

18. The method according to claim 17, further comprising:
in response to receiving an access request for accessing the first privacy data from the second providing device, forwarding the access request to the first providing device, wherein the first providing device sends the first privacy data to the first electronic device after verifying the second providing device; and
receiving the first privacy data from the first providing device, and
forwarding the first privacy data to the second providing device.

19. The method according to claim 17, wherein in response to the first user selecting the first privacy data in a user interface of the first electronic device and correspondingly selecting, as an object to share the first privacy data, the second providing device from a plurality of providing devices displayed on the user interface, it is determined that the first privacy data is authorized to be shared with the second providing device.
